(19) United States
(12) Patent Application Publication
(10) Pub. No.: US 2001/0046302 A1
(43) Pub. Date: Nov. 29, 2001

(54) Active noise cancellation optimized air gaps

(76) Inventor: Paul D. Daly, Troy, MI (US)

Correspondence Address: Anthony P. Cho, Carlson, Gaskey & Olds. P.C., 400 W. Maple Rd., Ste. 350, Birmingham, MI, 48009, US

(21) Appl. No.: 09/798,220

(22) Filed: Mar. 2, 2001

Related U.S. Application Data
(60) Provisional application No. 60/197,138, filed on Apr. 14, 2000.

(51) Int. Cl. A61F 11/06; G10K 11/16; H03B 29/00

(52) U.S. Cl. 381/71.7; 381/71.5

(57) ABSTRACT

The air induction system comprises an air induction body having a mouth emanating a first sound and a speaker emanating a second sound disposed about the mouth. The mouth has at least a first mouth portion and at least a second mouth portion wherein the first sound is louder at the at least first mouth portion than at the at least second mouth portion. The speaker has at least a first speaker portion and at least a second speaker portion wherein the second sound is louder at the at least first speaker portion than at the at least second speaker portion.

This application claims priority to Provisional Patent Application Serial No. 60/197,138 filed Apr. 14, 2000.

### BACKGROUND OF THE INVENTION

This invention relates to an active control of automotive induction noise.

Manufacturers have employed active and passive methods to reduce engine noise within the passenger compartment. Such noise frequently emanates from the engine, travels through the air induction system and emanates out of the mouth of the air intake into the passenger compartment. Efforts have been made to reduce the amount of engine noise traveling through the air induction system. These efforts include the use of both passive devices such as expansion chambers and Helmholtz resonators and active devices involving anti-noise generators.

Active systems use a speaker to create a sound that attenuates engine noise. The sound created is out of phase with the engine noise and combines with the engine noise to result in its reduction. Generally, this sound is generated in proximity to the air induction system. In one such system, the speaker is placed in the mouth of air intake duct.

Air ducts frequently have a non-round mouth such as an ellipse shape. In such an instance, the speakers used in such systems may also have a non-round shape to conform to the shape of the mouth of the air duct. Non-round speakers, however, have varying levels of sound generation quality due to their shape. That is, a non-round speaker will create a louder sound in some portions of the speaker than in other portions because of its dimensions. For example, a speaker in the shape of an ellipse will radiate sound louder at the points of the ellipse than on the sides of the ellipse. Due to this uneven sound production, the use of a non-round speak may result in a less than optimal attenuation of engine noise emanating from the mouth of the air induction system.

A need therefore exists to attenuate engine noise using a non-round speaker about a non-round mouth of an air induction system to accommodate for the speaker's uneven sound production.

### SUMMARY OF THE INVENTION

In a disclosed embodiment of this invention, the acoustic system comprises a mouth of an air induction body of an air induction system and a speaker disposed about the mouth. The mouth emanates a noise to be cancelled by a sound from the speaker. The mouth has at least a first mouth portion and at least a second mouth portion. The noise is louder at the first mouth portion than at the second mouth portion. The speaker generates a canceling sound and has at least a first speaker portion and at least a second speaker portion. The canceling sound is louder at the first speaker portion than at the second speaker portion.

The speaker is arranged about the mouth so that the first speaker portion, the louder speaker portion, is associated with the first mouth portion, the louder mouth portion. Preferably, the speaker is also arranged so that the second speaker portion is associated with the second mouth portion. The canceling sound is preferably out of phase with the engine noise to promote noise attenuation. In this arrangement, the more effective portion of the speaker is associated with the louder portion of the mouth to thereby improve engine noise attenuation.

If the mouth and speaker are elliptical in shape, the mouth is widened at portions closest to the louder portions of the speaker and narrowed at portions closest to the quieter portions of the speaker. Such a design also permits greater airflow through the air induction body than if the mouth and speaker had a uniform shape. Using this structure, other non-round speaker shapes can be optimized with an air duct mouth. In addition to attenuating engine noise, the invention may serve to amplify sound from a source. In such an arrangement, the sound from the speaker is in phase with the sound from the mouth and the louder portions of the speaker are associated with the louder portions of the mouth.

Both noise attenuation and sound amplification may be accomplished by similar structure. Accordingly, a first sound emanates from a mouth with at least a first mouth portion and at least a second mouth portion wherein the first sound is louder at the first mouth portion than at the second mouth portion. A second sound is generated from a speaker disposed about the mouth and has at least a first speaker portion and at least a second speaker portion wherein the second sound is louder at the first speaker portion than at the second speaker portion. The synchronization of the phase of sounds will determine whether the sounds are amplified or attenuated.

### BRIEF DESCRIPTION OF THE DRAWINGS

The various features and advantages of this invention will become apparent to those skilled in the art from the following detailed description of the currently preferred embodiment. The drawings that accompany the detailed description can be briefly described as follows:

FIG. 1 shows a side view of a speaker in the mouth of an air induction system as known in the art.

FIG. 2 shows a front view of FIG. 1, including the speaker and the mouth of the air induction system, as known.

FIG. 3 shows a front view of an embodiment of the invention, including elliptical speaker and an elliptical mouth of the air induction system.

FIG. 4 shows another view of the embodiment of FIG. 3.

FIG. 5 shows another embodiment of the invention, including another non-round speaker and non-round mouth.

### DETAILED DESCRIPTION OF THE PREFERRED EMBODIMENT

FIG. 1 illustrates a noise attenuation system known in the art. Shown are speaker 10, mouth 14, air induction body 18, air induction system 22 and engine 26. As known, air 18 travels through mouth 14, air induction body 18, and air induction system 22 to engine 26 to provide air for the engine's cylinders. Conversely, engine noise 34 travels from engine 26 through air induction system 22, air induction body 18, and out of mouth 14. Speaker 10 creates canceling sound 38, which is a sound wave that is out of phase with engine noise 34. It is known in the prior art that the face of speaker 10 is preferably in the same plane as mouth 14.

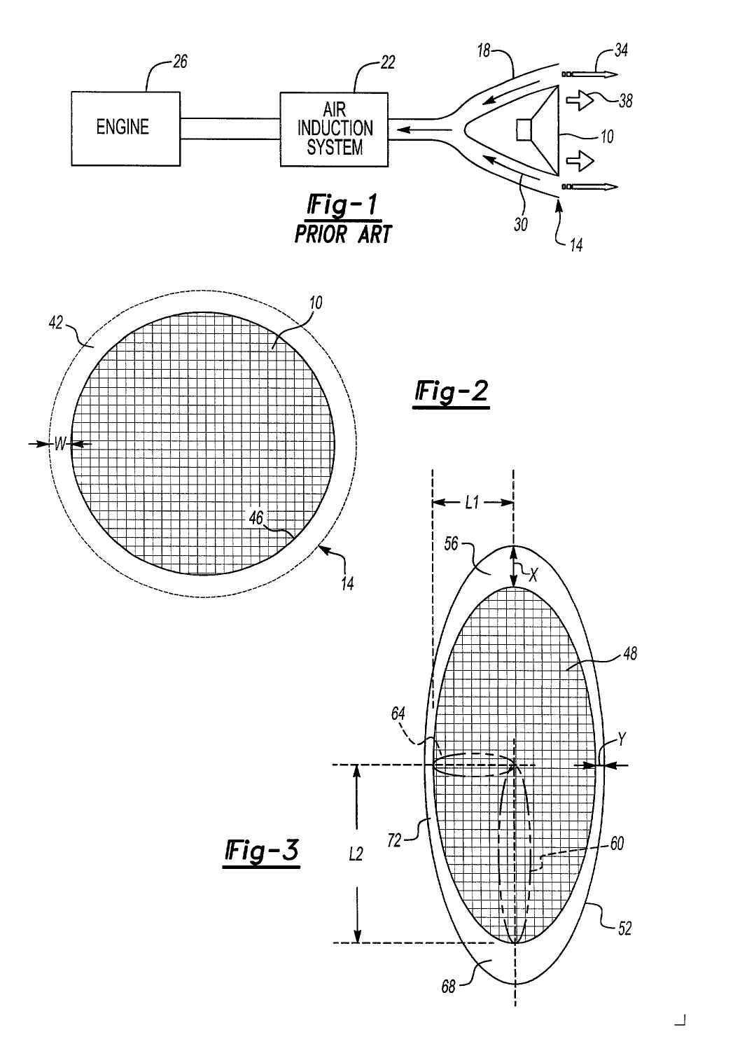

FIG. 2 represents a front view of speaker 10 and mouth 14 of FIG. 1. For a round speaker, speaker effectiveness is uniform around edge 46 of speaker 10. To promote optimal noise cancellation, gap 42 between speaker 10 and mouth 14 is therefore of uniform width W.

FIG. 3 illustrates an embodiment of the invention. Speaker 48, an ellipse, is shown disposed about mouth 52, another ellipse. Mouth 52 is operatively connected to an air induction system as shown in FIG. 1 and as known in the art. Because length L2 of speaker 48 is longer than L1, more air is compressed and sound generated at first speaker portion 60 along length L2 than at second speaker portion 64 along length L1. Thus, speaker 48 has a higher sound level at first speaker portion 60 than at second speaker portion 64.

To maximize noise cancellation by speaker 48, gap 56 between mouth 52 and speaker 48 is of non-uniform width as illustrated at X and Y, where width at X is greater than width at Y. Accordingly, first mouth portion 68 and second mouth portion 72 comprise gaps of differing widths as measured between speaker 48 and mouth 52. This arrangement permits more engine noise to emanate from mouth 56 at first mouth portion 68 than at second mouth portion 72.

Moreover, first speaker portion 60 is associated with first mouth portion 68 and second speaker portion 64 is associated with second mouth portion 72. In this way, the louder portions of speaker 48 are associated with the louder portions of mouth 52 while the quieter portions of speaker 48 are associated with the quieter portions of mouth 52. Thus, the more effective portions of speaker 48 are placed by the louder portions of mouth 52 while engine noise is minimized around the quieter portions of speaker 48. As known, the sound from speaker 48 is preferably 180 degrees out of phase with engine noise to ensure destructive interference between the two sounds. Alternatively, if the sound from mouth 52 shares a similar phase to sound from speaker 48, the two sounds are thereby amplified rather than attenuated.

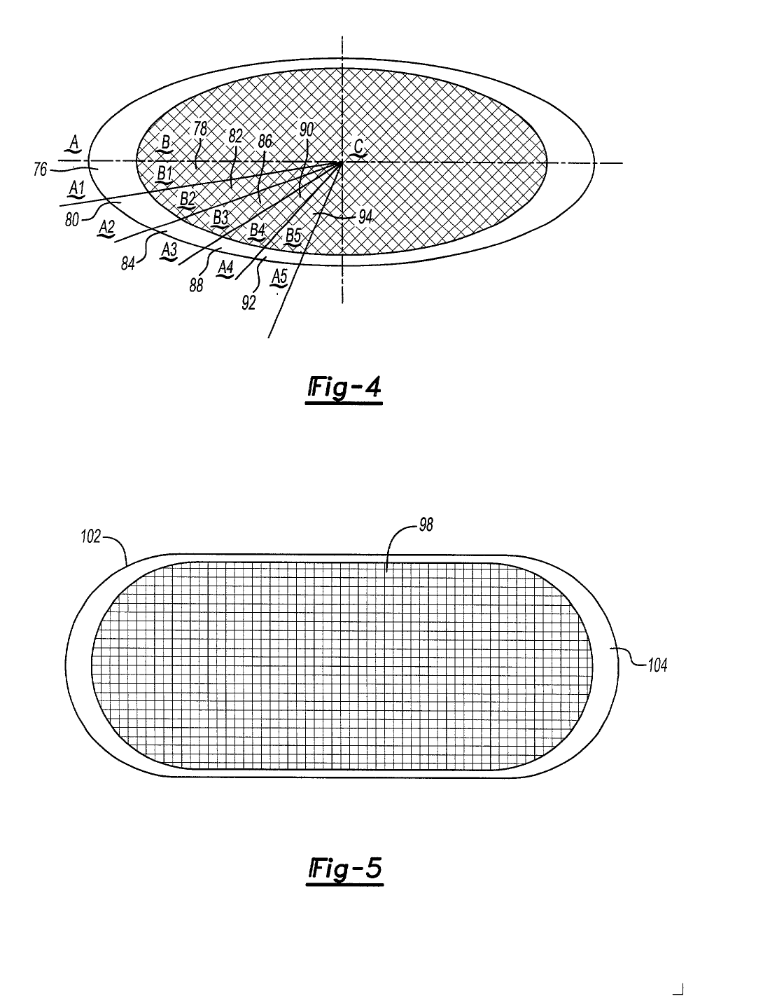

FIG. 4 illustrates a way to determine optimal spacing of gap 56 between speaker 48 and mouth 52. If area 76, a mouth portion, is delineated by points A, B, A1, and B1 and area 78, a speaker portion, is delineated by points B, C, B1, then the division of area 76 by area 78 creates a constant k. Constant k permits determination of the relationship between area 80 as delineated by points A1, B1, B2 and A2 and area 82 as delineated by points B1, C, and B2. This relationship is as follows:

Area 80 (A1, B1, B2, A2)/k=Area 82 (B1, C, B2)

Similarly, area 84 is related to area 86 is:

Area 84(A2, B2, B3, A3)/k=Area 86 (B2, C, B3)

Constant k will relate areas 88 and 90 and 92 to 94 in the same way.

Thus, k once known allows ideal determination of the spacing around the speaker. Moreover, as known in the art, constant k may be multiplied by a factor related to wavelength of sound and width of gap to compensate for attenuation of sound due to small gap effects.

FIG. 5 illustrates another embodiment of the invention using a non-round speaker 98 with a non-round mouth 102. Again, gap 104 between speaker 98 and mouth 102 is of non-uniform width, permitting more noise to emanate around the more effective portions of speaker 98.

In FIGS. 3-5, a first sound emanates from a mouth with at least a first mouth portion and at least a second mouth portion wherein the first sound is louder at the first mouth portion than at the second mouth portion. A second sound also emanates from a speaker disposed about the mouth and has at least a first speaker portion and at least a second speaker portion wherein the second sound is louder at the first speaker portion than at the second speaker portion. A person of ordinary skill in the art can employ this feature to develop and employ other shapes to thereby maximize speaker effectiveness in relationship to the sound emitted from the mouth to either attenuate or amplify sound.

The aforementioned description is exemplary rather then limiting. Many modifications and variations of the present invention are possible in light of the above teachings. The preferred embodiments of this invention have been disclosed. However, one of ordinary skill in the art would recognize that certain modifications would come within the scope of this invention. Hence, within the scope of the appended claims, the invention may be practiced otherwise than as specifically described. For this reason the following claims should be studied to determine the true scope and content of this invention.

What is claimed is:

1. An acoustic system comprising:
a mouth emanating a first sound and with at least a first mouth portion and at least a second mouth portion wherein said first sound is louder at said at least first mouth portion than at said at least second mouth portion; and
a speaker emanating a second sound disposed about said mouth and with at least a first speaker portion and at least a second speaker portion wherein said second sound is louder at said at least first speaker portion than at said at least second speaker portion.

2. The acoustic system of claim 1 wherein said speaker is arranged so that said at least first speaker portion is associated with said at least first mouth portion.

3. The acoustic system of claim 2 wherein said speaker is arranged so that said at least second speaker portion is associated with said at least second mouth portion.

4. The acoustic system of claim 1 wherein said speaker is arranged so that said at least second speaker portion is associated with said at least second mouth portion.

5. The acoustic system of claim 1 wherein said first mouth portion has a width between said speaker and said mouth different from a width between said speaker and said mouth of said second mouth portion.

6. The acoustic system of claim 5 wherein an area of said first mouth portion is related to an area of said first speaker portion so as to define a constant, said constant being used to relate said second mouth position to said second speaker position.

7. The acoustic system of claim 1 wherein said second sound is out of phase with said first sound to attenuate said first sound and said second sound.

8. The acoustic system of claim 1 wherein said second sound is in phase with said first sound to amplify said first sound and said second sound.

9. The acoustic system of claim 1 wherein said mouth is operatively connected to an air induction system.

10. An air induction system comprising:
an air induction body having a mouth emanating a first sound and with at least a first mouth portion and at least a second mouth portion wherein said at least first sound is louder at said at least first mouth portion than at said at least second mouth portion; and
a speaker emanating a second sound disposed about said mouth and with at least a first speaker portion and at least a second speaker portion wherein said second sound is louder at said at least first speaker portion than at said at least second speaker portion and wherein said second sound is out of phase with said first sound.

11. The air induction system of claim 1 wherein said speaker is arranged so that said at least first speaker portion is associated with said at least first mouth portion.

12. The air induction system of claim 11 wherein said speaker is arranged so that said at least second speaker portion is associated with said at least second mouth portion.

13. The air induction system of claim 10 wherein said speaker is arranged so that said at least second speaker portion is associated with said at least second mouth portion.

14. The air induction system of claim 1 wherein said first mouth portion has a width between said speaker and said mouth different from a width between said speaker and said mouth of said second mouth portion.

15. The air induction system of claim 15 wherein said first mouth portion is related to an area of said first speaker portion so as to define a constant, said constant being used to relate said second mouth position to said second speaker position.

16. The acoustic system of claim 10 wherein said mouth is operatively connected to an air induction system.

17. A method of attenuating sound comprising the steps of:
emanating a first sound from a mouth of an air induction body with at least a first mouth portion and at least a second mouth portion wherein the first sound is louder at the at least first mouth portion than at the at least second mouth portion; and
emanating a second sound from a speaker disposed about the mouth and with at least a first speaker portion and at least a second speaker portion wherein the said second sound is louder at the at least first speaker portion than at the at least second speaker portion and wherein the second sound is out of phase with the first sound.

18. The method of claim 17 further wherein the speaker is arranged so that the at least first speaker portion is associated with the at least first mouth portion.

19. The method of claim 18 wherein the speaker is arranged so that the at least second speaker portion is associated with the at least second mouth portion.

20. The method of claim 17 wherein the speaker is arranged so that the at least second speaker portion is associated with the at least second mouth portion.
